(19) United States
(12) Patent Application Publication
(10) Pub. No.: US 2009/0293165 A1
(43) Pub. Date: Dec. 3, 2009

(54) VISOR OVERLAY ASSEMBLY

(76) Inventor: Derek Leslie ARNOLD, Emmeloord (NL)

Correspondence Address: HOWREY LLP-EU, C/O IP DOCKETING DEPARTMENT, 2941 FAIRVIEW PARK DR., SUITE 200, FALLS CHURCH, VA, 22042, US

(21) Appl. No.: 12/538,207

(22) Filed: Aug. 10, 2009

Related U.S. Application Data
(63) Continuation of application No. PCT/GB08/50081, filed on Feb. 8, 2008.

(30) Foreign Application Priority Data
Feb. 9, 2007 (GB) 0702505.9

(51) Int. Cl. A61F 9/00

(52) U.S. Cl. 2/15

(57) ABSTRACT

A visor assembly according to various aspects of the present invention includes a visor having a surface; an overlay-sheet; and a mechanical fastening for attaching the overlay-sheet to the surface of the visor. The mechanical fastening includes a first fastening portion provided on the surface of the visor, an array of projections upstanding from the visor surface; and a second fastening portion provided on the overlay-sheet, including an array of complementary projections upstanding therefrom. The projections of the first and second fastening portions are configured to inter-engage in order to attach the overlay-sheet to the visor.

### CROSS REFERENCE TO RELATED APPLICATIONS

This application is a continuation of international application no. PCT/GB2008/050081, filed on Feb. 8, 2008, and claims priority from United Kingdom application no. 0702505.9, filed on Feb. 9, 2007. The contents of both applications are hereby incorporated by reference in their entirety.

### BACKGROUND OF THE INVENTION

1. Field of the Invention

The invention relates generally to visor assemblies which comprise an overlay-sheet and a visor releasably attached to one another. More particularly the invention relates to such visor assemblies for motorbike style helmets such as quad-bike, snowmobile, racing car and skiing helmets; heavy-duty protective visors, for example, ballistic face shields which may be used in riot helmets and visors for use by the emergency services; and goggles such as underwater diving goggles, motorcycle goggles or skiing goggles. Windows in vehicles and instrument covers exposed to the open air, and the like, can also make use of the technology according to the invention.

2. Description of the Related Art

Visor assemblies comprising a visor with an overlay-sheet releasably attached thereto by mechanical fastenings, are generally known.

In such visor assemblies the visor is more substantial than the overlay-sheet and acts as a face shield. In the case of motorbike style helmets the visor acts to protect the user's face from wind, rain, dirt and grit; and in the case of ballistic visors it acts to protect the user's face from more substantial projectiles and blows. In goggles the visor tends to be limited to extending over the eyes and that part of face immediately adjacent the eyes. The visor of the goggles may have different functions depending on usage. For example diving goggles are worn to aid underwater vision, motorcycle goggles are worn to protect a user's eyes from projectiles and dirt, and ballistic goggles are worn to protect a user's eyes from more substantial projectiles. Goggles may be provided with two visors, one per eye.

Visors may be provided in 1-dimensional (1-D) form, that is occupying a single plane so as to be flat; 2-dimensional (2-D) form, that is curved in one direction; or 3-dimensional (3-D) form, that is curved in two directions so as to be bowled. Overlay-sheets may be provided in appropriate shapes to fit to the surfaces of these various visor forms. In this respect, overlay-sheets may also be 1-D, 2-D or 3-D. 1-D overlay sheets are used with 1-D visors; 1-D and 2-D overlay-sheets are used with 2-D visors; and 3-D overlay-sheets are used with 3-D visors.

The overlay-sheet is typically utilized to provide an improved viewing window for the visor wearer. For example, the overlay-sheet may be configured to have an anti-condensation function to prevent misting-up of the viewing area. The overlay-sheet may also be provided with tinting to give improved viewing in varying light conditions. Examples of helmet visor assemblies are known from U.S. Pat. Nos. 5,765,235 and 6,922,850, the contents of which are hereby incorporated by reference in their entirety, which provide anti-condensation overlay-sheets attached to visors. In these applications the visors are provided with mechanical retaining means for retaining an inner overlay-sheet. The retaining means takes the form of pin-shaped elements against which the overlay-sheet abuts. Recesses are provided in the overlay-sheet which engage with the pins to provide a secure retention on the inside of the curved visor.

Such a system offers excellent retention of an inner overlay-sheet but suffers from the disadvantage that the mechanical retaining means in the form of the pin-shaped elements, must be fitted through openings in the visor. The provision of such openings requires additional processing at a manufacturing stage if the visor is manufactured for use with an overlay-sheet; or it requires skilled and accurate adaptation of a pre-existing visor in the event of retrofitting of an overlay-sheet. In both cases the situation is further complicated by considerations of how the provision of the required holes may affect the structural integrity of the visor. The integrity of the visor is of major importance, in particular in order to meet various governmental safety requirements which visors are often subjected to.

In order for such visor combinations to function effectively, the position of the mechanical retainers is critical in order to effectively tension the overlay-sheet into engagement with the visor. Any play between the components can lead to friction, irritating noise and abrasion e.g. of the viewing region of the visor. In the particular case of a visor assembly in which it is intended to form a sealed chamber between the overlay-sheet and the visor, movement of the overlay-sheet can result in loss of the seal thus allowing ingress of moisture and dirt to the chamber. If holes are drilled in order to retro-fit an overlay sheet, any inaccuracy may cause leakages by the pins or render the whole assembly to be useless. Attempts have been made to improve retention by the use of eccentric pins that may be tightened by rotation, for example, through 180°.

Easy fitting of a mechanical retention system during manufacture or post-manufacture of a visor would be of great benefit.

The pin and recess visor assembly of the state of the art is suitable only for use with a visor assembly in which the overlay-sheet is provided on the inner-surface of a curved visor. This is because the pin and recess system of these state of the art systems acts as a retaining means by providing a compressive tension to the overlay-sheet so that the overlay-sheet is pushed toward the inner surface of the visor.

In some circumstances it is advantageous to provide the overlay-sheet on the outer surface of a curved visor, or to provide the overlay-sheet on a flat visor. For such constructions a retaining mechanism must act to pull the overlay-sheet towards the visor surface. In the case of a flat visor this is achieved by providing a connection force acting directly toward the visor surface. In the case of a curved visor this is achieved by providing a laterally outward force with respect to the overlay-sheet so that the overlay-sheet is pulled around the outer surface of the visor.

State of the art fastening mechanisms are specific to their particular use so that the same mechanism cannot be used for the alternatives of fastening to inner and outer surfaces of a curved visor; or to a flat visor.

A fastening mechanism usable in all three situations would be of great advantage from the point of view of manufacture and use.

The overlay-sheet of such visor assemblies is preferably biased toward the visor when attached thereto. This is particularly of benefit where a seal member is provided between the overlay-sheet and the visor since a compressive strain results on the seal member ensuring an effective air tight seal.

The biasing of the overlay-sheet is achieved in the state of the art pin and recess system by a compressive action on the overlay-sheet. The biasing of an overlay-sheet attached to the outer surface of a visor is achieved by stretching of the overlay-sheet around the visor surface.

Visors generally vary in their thickness and hence flexibility according to the use for which they are designed. For example, a motorbike helmet visor need only be thick enough to deflect fairly light weight projectiles, its thickness being kept to a minimum in order to reduce weight. Motorbike helmet visors are therefore quite flexible. A ballistic visor, however, must be thick and stiff enough to withstand impacts from heavier projectiles. A ballistic visor is therefore generally less flexible than a motorbike helmet visor. This causes problems when using the pin and recess mechanisms discussed above because in order to fit the overlay-sheet the visor must initially be flexed at least partially out of its curved state. This moves the pins away from one another and allows the recesses of the overlay-sheet to be engaged with the pins before they are allowed to move back to their original positions locking the overlay-sheet in place. The lack of flexibility in the ballistic visor makes single-handed fitting of the overlay-sheet with this retention system virtually impossible. Attempts have been made to overcome this problem by the use of eccentric pins which can be rotated into and out of engagement with the overlay sheet recesses.

Fitting of overlay-sheets to in-flexible visors, for example visors made of glass, as in glass goggles or instrument panels; or 3-D visors, also need to have retaining means which can be utilized with minimal flexing of the visor or without any flexing of the visor.

### BRIEF SUMMARY OF THE INVENTION

According to the present invention there is provided a helmet visor assembly comprising, a visor, an overlay-sheet; and a mechanical fastening for attaching the overlay-sheet to the visor; wherein the mechanical fastening comprises a first fastening portion on the visor, comprising an array of projections upstanding from the visor surface; and a second fastening portion provided on the overlay-sheet, comprising an array of complementary projections upstanding therefrom; wherein the projections of the first and second fastening portions are adapted to inter-engage in order to attach the overlay-sheet to the visor.

A mechanical fastening having arrays of complementary projections adapted to inter-engage with one another provides an adequately secure connection to hold the overlay sheet in attachment with the visor without requiring the provision of holes for fitting the mechanical fastening to the visor. It also provides a secure non-permanent connection so that the overlay-sheet can be releasably attached to the visor. This advantageously allows easy removal or replacement of the overlay sheet.

Preferably when fitted the overlay-sheet is spaced from the visor by one or more spacer elements arranged between the visor and the overlay-sheet. In a particularly preferred embodiment the spacer element may take the form of a seal element which acts to form a sealed compartment between the overlay-sheet and the visor.

In a preferred embodiment the first and second fastening portions comprise similar arrays of projections; that is, the arrays comprise the same general spacing, density and size of projections as each other. For the sake of clarity, the fastening portions or the arrayed areas need not be the same size as their counterparts.

The fastening portions for motorbike style visors, ballistic visors or goggle visors are preferably located on the short, vertical (during use) edges of the overlay sheet.

In a particular embodiment the fastening portions comprise a backing having on one side an adhesive layer and on an opposed side the array of projections integrally formed therewith and upstanding therefrom. The fastening portions are secured to the visor or the overlay-sheet by the adhesive layer. The adhesive layer is preferably a pressure sensitive adhesive and/or comprises an acrylic base. It is preferred that the base is non-aggressive, non-corrosive and semi-permanent.

Providing the fastening portions on a backing with adhesive, allows the fasteners to be either fitted to the components of a visor assembly at the point of manufacture without the provision of holes, or to be easily retrofitted to visors. Such retrofitted visors may not necessarily have been manufactured for use in a visor assembly of the present sort. This latter advantage is of particular benefit since it allows a consumer to adapt their pre-existing visor for use with an overlay-sheet but without the difficulties of accurately drilling holes or the concerns that the drilling of such holes may affect the structural integrity of the visor.

One or both of the first and second fastening portions may be greater in area than strictly necessary to provide the required holding force. This advantageously allows some adaptation of the location of the overlay-sheet even after provision of the fastening portions on the visor and the overlay sheet. It is only necessary to have a sufficient overlap of the two portions in order to provide the required holding force so the overlay-sheet can have different locations on the visor. This advantageously means that it is possible to reposition the overlay-sheet even after fitting is completed. Such a repositioning is not possible with the pin and recess system of the state of the art, which provides a single position fastening mechanism and hence requires very accurate fitting with specialist equipment. With a multiposition fastening mechanism a level of inaccuracy in fitting of the mechanism can be tolerated because by repositioning of the overlay-sheet on the fastening portions of the visor the correct position of the overlay-sheet can be attained.

One aspect of the invention is realized in the form of a kit of parts for constructing the above visor assembly. Such a kit of parts comprises an overlay-sheet adapted to be fitted to a surface of a visor; a first fastening portion associated with the overlay-sheet, for attaching the overlay-sheet to the visor; wherein the first fastening portion comprises a fastening element having an array of upstanding projections; and a second fastening portion comprising a backing having an array of projections upstanding from one side and on an opposed side an adhesive; wherein the first and second fastening portions are adapted to inter-engage in order to provide a mechanical fastening.

In this manner an overlay-sheet can be provided to a consumer owning a pre-existing visor and the overlay-sheet fitted merely by sticking fastening tabs to the visor appropriately.

The fastening portion associated with the overlay-sheet may be provided with a fastening tab for application by the consumer, or may be pre-adhered to the overlay-sheet. Alternatively the fastening portion may be integrally formed with the overlay-sheet.

In a preferred embodiment a template of paper, carton or anti-static film may be provided along with the kit of parts to aid the positioning of the tabs on the visor and/or the overlay-sheet. Alternatively, a shallow shaped recess or a number of such recesses, preferably having a depth slightly less deep than that of the fastening tabs, can be provided in the visor and/or overlay-sheet. The recess or recesses may be provided, for example, by milling. Depending upon the particular visor assembly application, such recesses may be provided on the internal surface, external surface or both internal and external surfaces of the visor. The recess or recesses and the fastening tab or tabs are advantageously adapted to have complementary dimensions to one another. This enables accurate fitting of the tabs because the recesses denote where the tabs should be mounted on the visor and/or the overlay-sheet. The provision of such recesses removes the need for a separate template and enables quick, easy and accurate fitting of the tabs. A visor provided with such a recess or recesses is pre-prepared for retrofitting of an overlay-sheet. Advantageously, such a pre-prepared visor can be supplied to a consumer without the fastening portions, thus reducing the cost of the visor, but can be easily adapted for fitting of an overlay-sheet post-sale.

The projections are preferably formed from thermoplastic resin and have a molecular orientation of at least 0.001 as evidenced by their birefringence value. The molecular orientation of the projections results in good stiffness, durability, tensile strength and flexural strength.

The birefringence value is calculated using an “Ortholux 2 Pol” polarized light microscope with a Berek compensator from E. Leitz Company, Covington, Ky. A tab of the projections is placed under crossed polarized light with its z-axis oriented north-south. The microscope stage is rotated 45 degrees. A compensator is rotated in each direction until a black fringe appears; at this point retardations are equal and opposite. Compensator readings are recorded and the birefringence of the sample is calculated according to the equation:

B=R×C/t

where R=retardation, C=compensator constant, and t=sample thickness. The retardation R is defined as the phase difference between the two components in numbers of waves.

Examples of suitable thermoplastic resins from which the projections can be formed include polyesters, such as poly(ethylene terephthalate); polyamides such as nylon, poly(styrene-acrylonitrile), poly(acrylonitrile-butadiene-styrene); polyolefins such as polypropylene, and plasticized polyvinyl chloride.

The density of the projections on the fastening portions is preferably from 60 to 1,550 projections per square cm, more preferably from about 90 to 690 projections per square centimeter and most preferably about 100 projections per square centimeter.

The array of projections is laid out such that lateral movement of two inter-engaged fastening portions is limited. This is important because it has been discovered that lateral slippage of the fastening portions over one another can lead to poor anchoring of the overlay-sheet. This is particularly problematic where the overlay-sheet is an anti-condensation sheet provided with a sealed air chamber between the overlay-sheet and the visor. This sealed air chamber creates an insulating gap to prevent misting of the viewing area of the visor. Poor anchoring in such a construction can lead to a poor seal and subsequent ingress of external air, moisture and dirt into the sealed chamber between the overlay-sheet and the visor. Therefore a good anchoring by the mechanical fasteners in relation to lateral slippage is particularly important when an airtight seal is to be provided.

Lateral movement can be limited by providing the array of projections in an unordered or non-linear lay-out. In one such arrangement the positions of the individual projections are mathematically unpredictable except that minimum and maximum spacing between adjacent projections are determined. Such a lay-out is discussed in U.S. Pat. No. 3,408,705, the entirety of which is hereby incorporated by reference.

An alternative method of preventing lateral slippage is found in the provision of the projections in rows, wherein the rows follow a wave-like path. Such a system is described in U.S. Pat. No. 540,275, the entirety of which is hereby incorporated by reference.

As a consequence of the fact that lateral slippage is prevented, the overlay-sheet may be held either in tension or in compression. Such a device is thus suitable for use on either an inner or an outer surface of a 2-D or 3-D visor; or on a 1-D visor.

In one embodiment the fastening portions are formed integrally with one or both of the visor and the overlay-sheet.

In a particularly preferred embodiment of the present invention a plurality of the projections comprise stems, each stem having a mushroomed portion along its length. An array of mushroom shaped projections results. Such mushroom shaped projections exhibit good interengagement and therefore provide an excellent connection of the overlay-sheet to the visor.

Preferably the mushroomed portion takes the form of a section overhanging the stem on all sides.

A suitable tab of mushroom shaped projections is the fastener sold under the trade name Dual Lock™ by 3M™.

Methods of manufacturing suitable tabs and mushroom-shaped projections are disclosed in United States patent application No. 20030689111 and European Patent application No. 19940912756, hereby incorporated by reference in their entirety.

As briefly discussed above, the overlay-sheet may be provided with a sealing member. This sealing member acts to space the overlay-sheet from the visor and forms a sealed compartment therebetween. This sealed compartment acts as an insulator reducing the possibility of condensation formation in the viewing area of the visor.

Preferably the seal member is adhered to the overlay-sheet and is held in non-adhesive relation to the visor by the mechanical fastening so that it forms an airtight seal but does not adhere to the visor. Since the seal member is not adhered to the visor the overlay-sheet is removable from the visor so that it can be replaced if damaged, or removed or replaced depending upon weather conditions.

The seal member is preferably made of silicone material, particularly transparent silicone material. This acts as a flexible seal between the overlay-sheet and visor. Moreover, the compressive strain that results because of a biasing of the overlay-sheet toward the visor is uniformly distributed. Preferably the silicone material is dry, set and flexible silicone material but the skilled person will be well aware of other alternatives that may be considered equivalent to this material. The ingress of moisture and the like between the two shields is prevented as far as possible by means of such a construction. Because the seal member is not adhered to the visor, some movement relative thereto is possible. This is of importance if the overlay-sheet and the visor are made of different materials. One example of this is if the visor is made of polycarbonate and the overlay-sheet of cellulose acetate. Differences in both contraction and expansion can be overcome without any problem with the aid of a rubbery elastic seal. If cellulose acetate, cellulose propionate or other plastics are used it can be important to subject these to a heat treatment (tempering) in advance. The mechanical properties can be improved by this means. This relates to the material having the same properties in all directions and the restriction of shrinkage when the material is subsequently subjected to high temperature. Cellulose acetate or cellulose propionate, for example can be subjected to heat treatment at approximately 25-80° C. for approximately two hours for this purpose. This process can be repeated if deemed necessary to further reduce expansion and contraction properties. The number of repetitions is dependent upon the particular material used.

It will be clear to those skilled in the art that the seal member may be provided at alternative locations on the overlay-sheet so long as it encompasses an adequate viewing area for the visor user. For example the overlay-sheet may be larger than the viewing area of the visor but the seal member located on the periphery of the viewing area, and thus not on the periphery of the overlay-sheet.

The distance between the overlay-sheet and the visor can be adjusted as desired to optimise the anti-misting properties of the assembly.

The use of an insulating air gap between the visor and the overlay sheet provides good anti-misting properties without the need for electric heating elements as has been suggested in the prior art. The provision of electric heating elements is disadvantageous and it is hence preferred that the visor assembly of the present invention lacks electric heating elements.

Similarly as discussed above in relation to the visor, in a preferred embodiment the overlay-sheet is provided with recessed portions configured to receive the fastening portions. The depth of the recesses allows the spacing of the overlay-sheet and the visor to be adjusted to its optimum without compromising the required thickness of the fastening portions. Where recesses are provided, the fastening portions may be provided as tabs, dimensioned to fit complementarily with the recessed portions.

The recessed portions may be provided in various locations or numbers, for example four recesses and matching tabs may be provided generally at the laterally outer sides of the overlay-sheet. For motorbike style visors, ballistics visors or goggle visors, the recessed portions are preferably provided along the short, vertical (during use) edges of the overlay-sheet.

For curved visors (2D and 3D), the overlay-sheet may be provided on the inner concave surface of the visor or on the outer convex surface of the visor in order to prevent condensation formation on either the outside or the inside surface of the visor. In a particular embodiment the visor may be provided with both an inner overlay-sheet and an outer overlay-sheet, for environments where misting of both inner and outer visor surfaces is likely to occur. This is particularly advantageous in the case where work in enclosed humid spaces may be expected. For example a ballistic visor, such as those worn by riot police or armed forces, may become misted on both outer and inner surfaces while waiting in a vehicle for orders to advance, or on entering a warm building after having been waiting in a cold environment.

An overlay-sheet to be attached to the concave inner surface of a visor is provided with material thickness which allows for the build up of tensioning without buckling of the material. An overlay-sheet to be attached to the convex outer surface preferably has a thinner material thickness and may advantageously have a slightly elastic stretch so that when stretched to connect with the fastening portions it sits tightly over the outer surface of the visor.

In one embodiment of the invention the overlay-sheet is configured so that it can be fitted to both the inner surface and the outer surface of a visor, for example on 1-D and 2-D visors. This advantageously means that a single type of overlay-sheet can be used as either an outer-overlay sheet or an inner overlay-sheet. In an advantageous embodiment the overlay sheet may be provided with fastening portions on both of its major surfaces so that it can be fitted to either the inner or outer surface of a visor. In addition the overlay sheet may be provided with spacer elements or seal elements on both of its major surfaces so that a single visor forms an air-gap whether placed on the outer surface or inner surface of a visor.

When such an overlay-sheet embodiment is provided as a kit of parts enough fastening portion tabs are provided in the kit so that adequate fastening portions can be added to the visor for both inner and outer fitting.

As discussed, the visor assembly of the present invention maintains a user's vision through the visor assembly. In relation to this the overlay-sheet is preferably provided with an anti-misting surface, for example, in the form of a surface having hydrophilic properties. The surface may be applied as a coating of a hydrophilic material. The coating is preferably a silicone based material which is applied by dip-coating. More preferably the overlay-sheet is also provided with an anti-misting surface on both of its major surfaces.

One advantage of providing the coating on both surfaces is found in that a single coated overlay-sheet can be located on either the inside of the outside surface of a 1-D or 2-D visor. Since the overlay-sheet is coated on both sides, a hydrophilic surface is presented on the outersurface of the overlay-sheet in whichever position it is placed. Hence a single overlay sheet can provide the hydrophilic anti-misting function whether placed on the inner or outer surface of a visor.

The overlay-sheet may also be provided with a colouring agent in the form of a permanent colouring or a photo-chromic UV reactive dye. This acts to reduce the ingress of excess light during, for example, sunny conditions, or to filter particular wavelengths of light.

The photo-chromic overlay-sheet may take the form of a 3 ply laminate comprising two layers of polycarbonate and a central lamination adhesive impregnated with a powdered photo chromic dye. The percentage content of the dye can be varied as required in order to alter the level of shading of the overlay sheet in the activated state.

The overlay-sheet may be provided with an anti-scratch coating, separately or in combination with an anti-misting surface, on either or both of it major surfaces.

The overlay-sheet is preferably comprised of cellulose propionate, which advantageously has a good light transmission.

Visor assemblies of the present invention may comprise 1-D, 2-D or 3-D visors and or overlay-sheets.

In a preferred embodiment of the invention the visor of the visor assembly is provided with a recess shaped to receive an overlay-sheet. The dimensions of the recess preferably correspond closely to the peripheral dimensions of the overlay-sheet. The depth of the recess is preferably such that when the overlay-sheet is inserted it seats substantially flush with the un-recessed part of the visor. In such an embodiment additional securing mechanism may be provided in the form a snap-fit rim or the like around at least a part of the recess periphery.

Fastening portions for a visor assembly, comprising a first member comprising a permanently magnetic portion; and a second member configured to engage the first member and comprising a permanently or non-permanently magnetic portion adapted to engage in order to attach the inner-visor to the outer-visor are potentially useable instead of the fastening portions of the present invention, however they are considered to be inferior.

### BRIEF DESCRIPTION OF THE DRAWINGS

Specific embodiments of the invention will now be described by way of non-limiting example only. The features and advantages of the invention will be further appreciated upon reference to the following drawings, in which:

FIG. 1 shows a motorcycle style helmet provided with a visor assembly;

FIG. 2 shows a perspective view of a detail of the visor assembly of FIG. 1;

FIG. 3 shows an array of mushroom shaped projections;

FIG. 4 shows two inter-engaged arrays of mushroom shaped projections;

FIG. 5 shows two inter-engaged arrays of mushroom shaped projections integrally formed with a visor and an overlay-sheet;

FIG. 6 shows a schematic cross-section through an overlay-sheet;

FIG. 7 shows a schematic cross-section through a visor assembly during attachment of an overlay-sheet to the internal surface of a visor;

FIG. 8 shows a schematic cross-section through a visor assembly during attachment of an overlay-sheet to the outer surface of a visor;

FIG. 9 shows a schematic cross-section through an overlay-sheet having recessed portions;

FIG. 10 shows a schematic cross-section through the overlay-sheet of FIG. 8 having a seal member;

FIG. 11 shows a schematic cross-section through a visor assembly during attachment of an overlay-sheet of FIG. 9;

FIG. 12 shows a visor assembly comprising a recessed visor;

FIG. 13 shows a cross-section along the line IV-IV of the visor of FIG. 12;

FIG. 14 shows a cross-section along the line V-V of the visor of FIG. 12;

FIG. 15 shows a visor assembly comprising a ballistic visor and having a first fastening arrangement;

FIG. 16 shows a visor assembly comprising a ballistic visor and having a second fastening arrangement; and

FIG. 17 shows a visor assembly comprising a ballistic visor and having a third fastening arrangement.

### DESCRIPTION OF ILLUSTRATIVE EMBODIMENTS

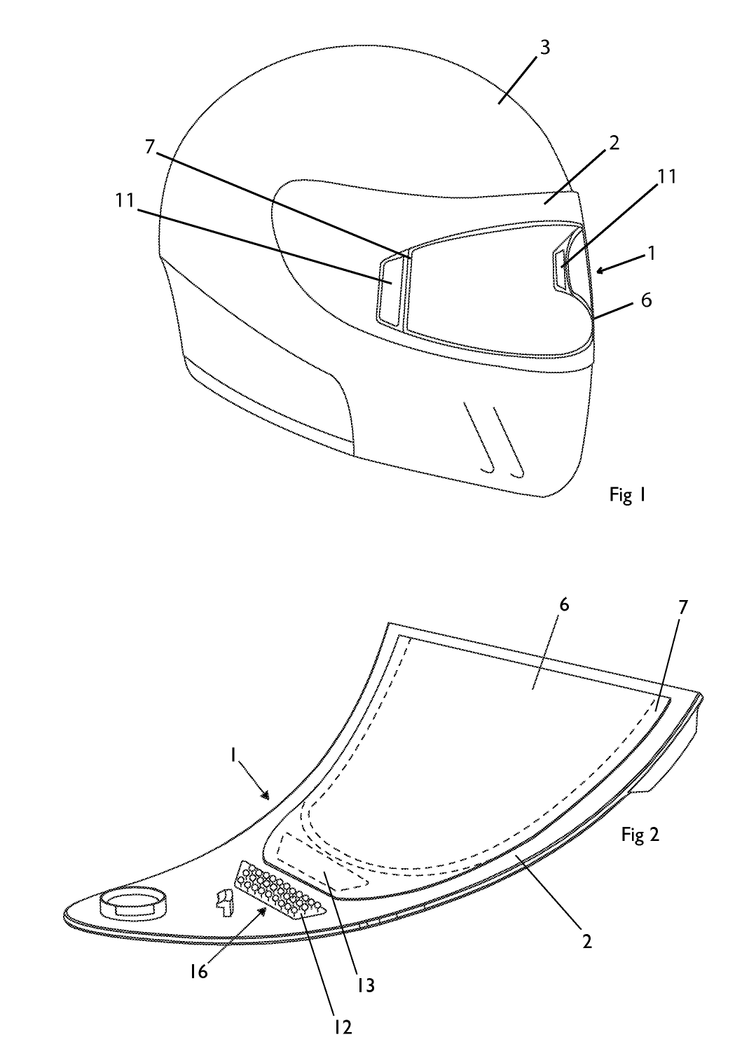

The following is a description of certain embodiments of the invention, given by way of example only and with reference to the drawings. FIG. 1 shows a motorcycle helmet 3 comprising a 3D visor assembly 1 in accordance with the present invention. There is provided a visor 2 having attached to its inner-surface an overlay-sheet 6. The overlay-sheet 6 is releasably attached to the visor 2 by a mechanical fastening 11.

The mechanical fastening 11 can be seen more clearly in FIG. 2 and is comprised of two hermaphroditic fastening portions 12, 13. The first fastening portion 12 is located on the visor 2 and the second fastening portion 13 is located on the overlay-sheet 6. The fastening portions 12, 13 are adapted to inter-engage with one another to attach the overlay-sheet 6 to the visor 2.

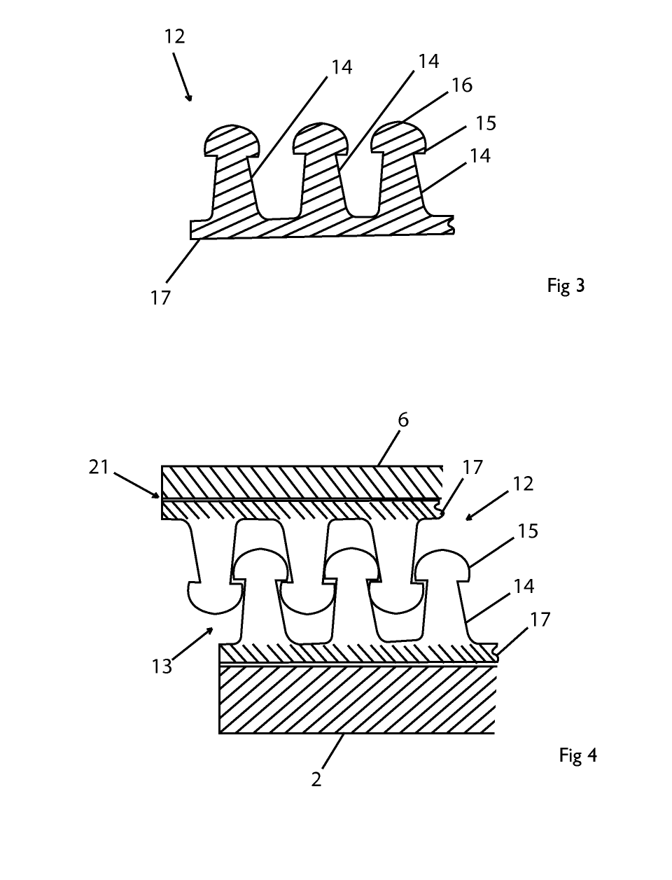

As illustrated in FIG. 3, each of the fastening portions is provided with an array of stems 14 upstanding therefrom. The stems 14 are each provided with a mushroomed portion 15 on their distal ends thus forming an array of mushroom-shaped projections 16.

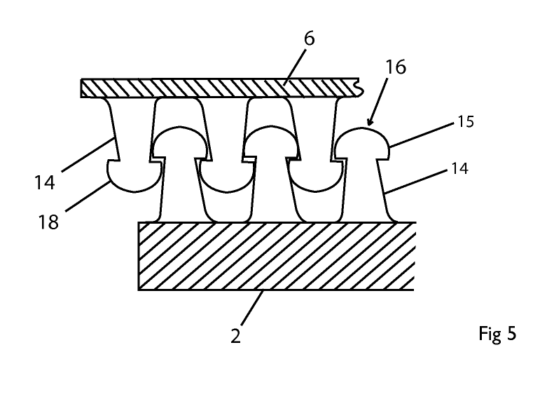

As shown in FIG. 4, the mushroom-shaped projections 16 are sized, spaced and provided in a density such that the arrays of the first and second fastening portions 12, 13 can inter-engage and form a connection therebetween. In this manner the overlay-sheet 6 is securely held in connection with the visor 2 during use.

In the embodiments illustrated in FIGS. 3 and 4 the arrays of mushroom-shaped projections 16 are provided integrally with a continuous backing 17 of thermoplastic resin. The backing of each portion is further provided with a layer of pressure-sensitive adhesive 21 with which it is attached to the visor 2 or the overlay-sheet 6.

An example of a suitable hermaphroditic fastener is the fastener tape sold under the tradename Dual Lock™ by 3M™.

Providing the fastening portions on a backing with adhesive, allows the fasteners to be either fitted to the components of a visor assembly 1 at the point of manufacture without the provision of holes which are required by other substantial enough mechanical connections, or can be retrofitted to visors. Such retrofitted visors may not necessarily have been manufactured for use in a visor assembly of the present sort. This latter advantage is of particular benefit since it allows a consumer to adapt their pre-existing visor for use with an overlay-sheet 6 but without the difficulties of drilling holes or the concerns that the drilling of such holes may affect the structural integrity of the visor.

In an alternative embodiment, the mushroom-shaped projections 16 may be integrally formed with one or both of the visor 2 or the overlay-sheet 6. This is preferably achieved by co-forming the projections 16 and either the visor 2 or overlay-sheet 6. In FIG. 5 the mushroom-shaped projections are integrally formed with the both the visor 2 and the overlay-sheet 6.

As illustrated in FIG. 1 a seal member 7 is provided around the periphery of the overlay-sheet 6. As a result of the presence of this seal the overlay-sheet 6 is spaced from the visor 2 and a compartment, sealed as far as possible with respect to the environment, is formed between the overlay-sheet 6 and the visor 2. This sealed compartment acts as an insulator reducing the possibility of condensation formation in the viewing area of the visor 2.

The seal member 7 is adhered to the overlay-sheet 6 and is held in non-adhesive relation to the visor 2 by the mechanical fastening 11 so that it forms an airtight seal but does not adhere to the visor 2. In this manner the overlay-sheet 6 is removable from the visor 2 so that it can be replaced if damaged, or removed or replaced depending upon weather conditions.

The seal 7 is preferably made of transparent silicone material which can be mounted by a computer directed robot with a plunger dispensing nozzle. It will be clear to those skilled the art that the seal member 7 may also be comprised of alternative materials, for example natural or synthetic rubber or flexible closed cell (air tight) foams.

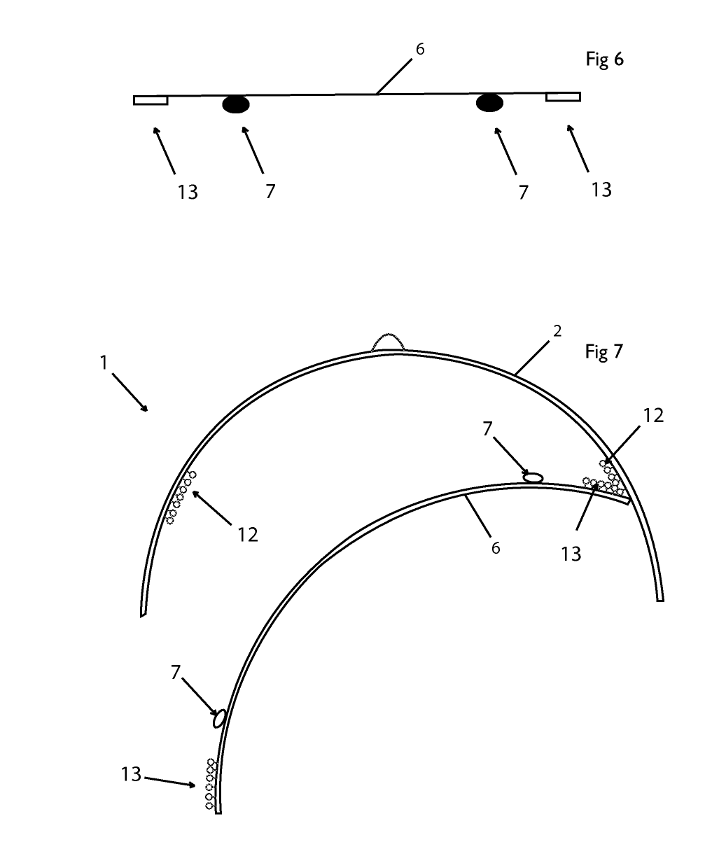

FIG. 6 shows a cross-sectional view through an overlay-sheet 6 provided with first fastening portions 13 on its periphery and seal member 7 at the periphery of the viewing area of the overlay-sheet.

The overlay-sheet 6 is fitted into the visor 2 as illustrated in FIG. 7.

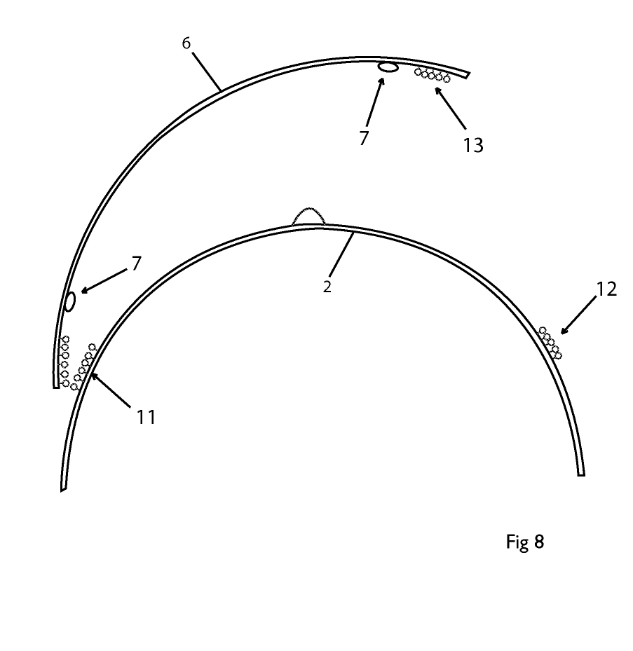

FIG. 8 illustrates the overlay-sheet 6 during attachment to the outer surface of the visor 2.

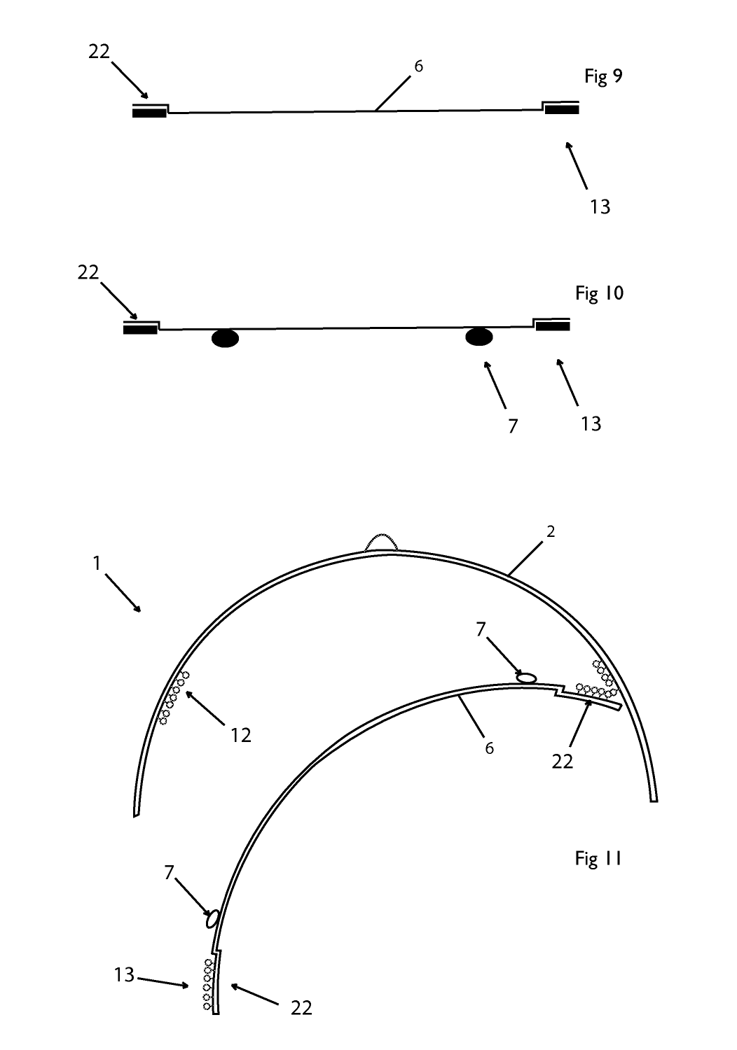

FIGS. 9 to 11 illustrate an embodiment of the invention in which the overlay-sheet is provided with recessed portions 22 in which the first fastening portions 13 are received. This has the advantage that, as can be seen from FIG. 11, the central portion of the overlay sheet is positioned more closely to the visor resulting in a shallower seal member 7 countering any increase in distance resulting from a thick fastening member. In this manner the spacing of the overlay-sheet from the visor can be adapted to its optimum without compromising on the thickness of the fastening mechanism.

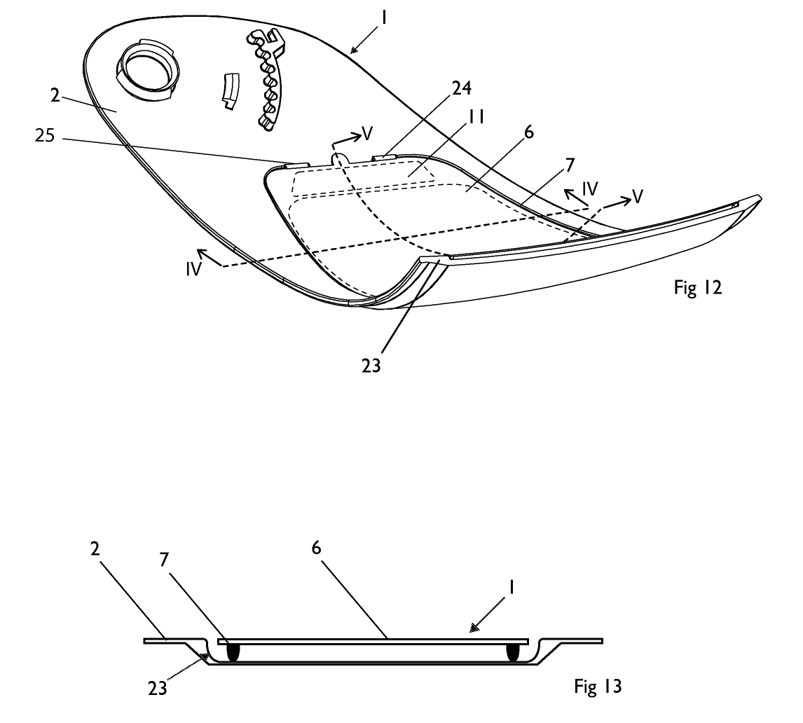

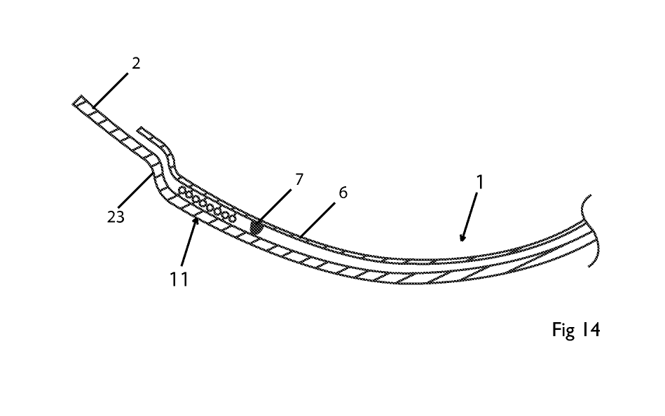

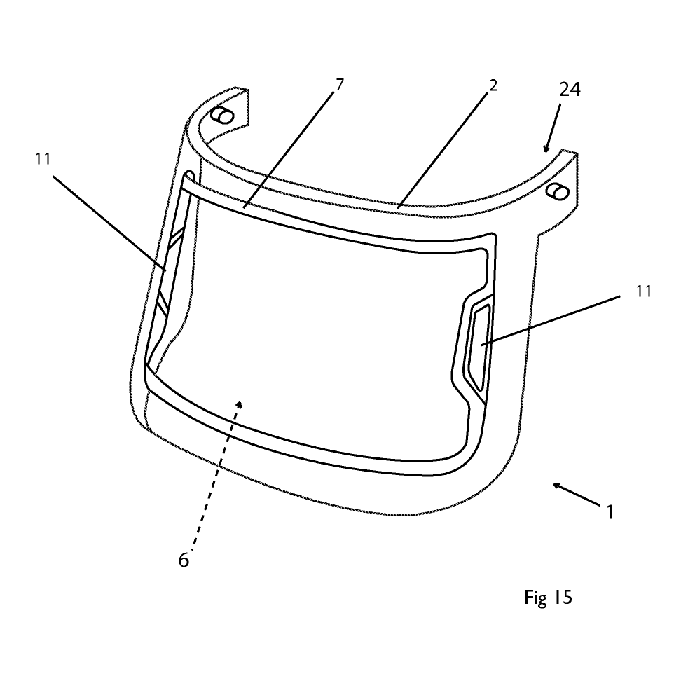

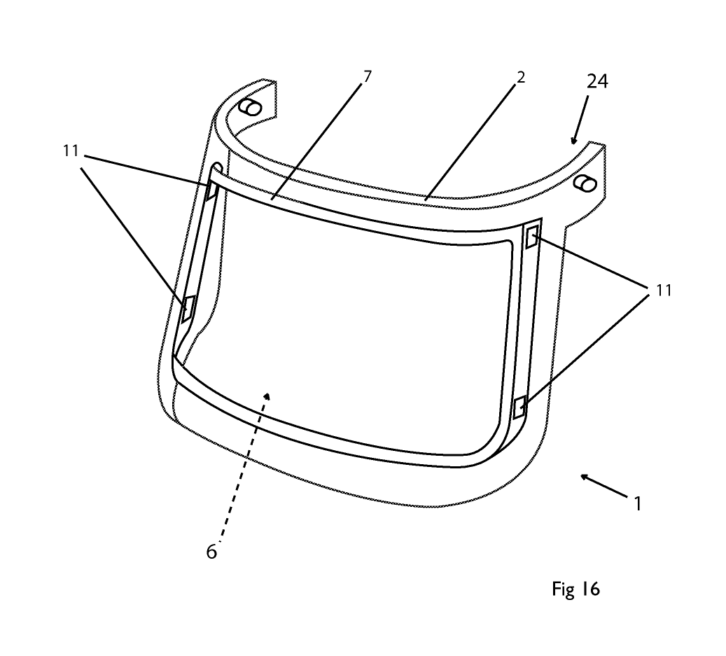

FIGS. 12, 13 and 14 show a visor assembly in which the visor 2 is provided with a recess 23. The dimensions of the recess correspond to the external dimensions of the overlay-sheet 6. As in the case of the embodiment of FIGS. 1 and 2 the overlay-sheet 6 is provided with a seal member 7 of flexible sealing material around its periphery. Fixing of the over-lay sheet 6 into the visor recess 23 is by the previously discussed fastening mechanism 11. The fastening is further aided by a snap-fit construction comprising snap-lips 24 and 25. This further pushes the overlay-sheet 6 against the visor 2 with some pretension. Seal member 7 provides a seal between the overlay-sheet 6 and the visor 2, as a result of which ingress of moisture, and consequently misting up of the visor 2, can be avoided.

FIG. 15 shows a ballistic visor 2 provided with an overlay-sheet 6 attached to its inner surface. The overlay-sheet 6 is provided with a silicone seal member 7 and is attached to the visor 2 by the mechanical fasteners 11 in the form of the arrays of mushroom-shaped projections illustrated in FIG. 4. Extended portions 24 are provided integrally with the visor and provided with apertures for connection to a helmet.

The ballistic visor is constructed more substantially than the visor of a motorcycle style helmet and typically has a thickness of between 4 mm to 15 mm (visors for motorcycle style helmets typically have a thickness of 3 mm or less). The overlay-sheet provided for use with the ballistic visor preferably has a thickness of between 0.2 mm and 1.15 mm for outer-overlay-sheets and between 0.6 mm and 1.15 mm for inner-overlay-sheets. Goggle visors are generally between 0.6 mm to 3 mm thick, the thinner visors being for Ski goggles or MotoX goggles and the thicker visors for ballistic purposes.

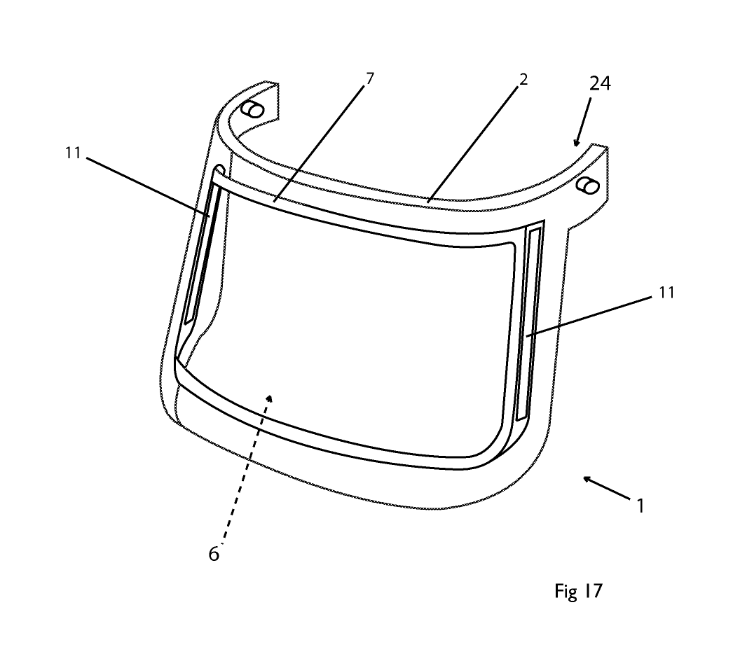

FIGS. 16 and 17 show similar ballistics visor assemblies to that of FIG. 15. In FIG. 16 the fastening portions 11 extend along essentially the whole height of the vertical ends of the visor. In FIG. 17 the fastening portions 11 are provided as four corner connectors.

In some instances Newton Rings may form on the overlay-sheet. In this event the thickness of the silicone seal member can be increased, thus increasing the distance between the two surfaces until the Newton Rings are sufficiently reduced or cleared.

All features discussed in relation to alternative visor types may be utilised in combination with the other mentioned visor types discussed in the introductory portion unless otherwise expressly stated.

Many modifications in addition to those described above may be made to the structures and techniques described herein without departing from the spirit and scope of the invention. Accordingly, although specific embodiments have been described, these are examples only and are not limiting upon the scope of the invention.

It should be noted that the term “comprising” as used in the claims or description of this application does not exclude other elements or steps; and the terms “a” and “an” do not exclude a plurality.

Equivalents and modifications not described above may also be employed without departing from the scope of the invention, which is defined in the accompanying claims.

What is claimed is:

1. A visor assembly, comprising:
a visor having a surface;
an overlay-sheet; and
a mechanical fastening for attaching the overlay-sheet to the surface of the visor;
wherein
the mechanical fastening comprises a first fastening portion provided on the surface of the visor, comprising an array of projections upstanding from the visor surface; and
a second fastening portion provided on the overlay-sheet, comprising an array of complementary projections upstanding therefrom; and
wherein the projections of the first and second fastening portions are adapted to inter-engage in order to attach the overlay-sheet to the visor.

2. The assembly of claim 1 wherein each projection comprises a stem having a mushroomed portion along its length.

3. The assembly of claim 1 wherein the projections are present on each fastening portion at a density of at least 60 projections per square centimeter.

4. The assembly of claim 1 comprising a seal member spacing the overlay-sheet from the visor and forming a sealed compartment between the visor and the overlay-sheet.

5. The assembly of claim 1 wherein the projections are composed of a plastic resin and have a molecular orientation as evidenced by a birefringence value of at least 0.001.

6. The assembly of claim 1 wherein the overlay-sheet is provided with a hydrophilic surface.

7. The assembly of claim 1 wherein the overlay-sheet is provided with tinting.

8. The assembly of claim 1 wherein the overlay-sheet is provided with an anti-scratch surface.

9. The assembly of claim 1 wherein the overlay-sheet comprises a recessed portion for receiving the first fastening portion.

10. The assembly of claim 1 wherein the visor comprises a recessed portion for receiving the second fastening portion.

11. The assembly of claim 1 wherein the projections of the first fastening portion are integral with the visor.

12. The assembly of claim 1 wherein the projections of the second fastening portion are integral with the overlay-sheet.

13. The assembly of claim 1 wherein two overlay-sheets are provided, a first attached to an inner surface of the visor and a second attached to an outer surface of the visor.

14. The assembly of claim 1 wherein the overlay-sheet is at least one of removable and repositionable.

15. A kit of parts comprising;
an overlay-sheet configured to be fitted to a surface of a visor; and
a first fastening portion associated with the overlay-sheet, for attaching the overlay-sheet to the visor; wherein
the first fastening portion comprises a fastening element having an array of upstanding projections; and
a second fastening portion comprising a backing having an array of projections upstanding from one side and on an opposed side an adhesive; and
the first and second fastening portions are configured to inter-engage in order to provide a mechanical fastening.

16. The kit of parts according to claim 15, wherein the first fastening portion comprises a backing, the array of projections upstanding from one side thereof and on an opposed side an adhesive.

17. An overlay-sheet configured to be fitted to a surface of a visor, comprising a mechanical fastening for attaching the overlay-sheet to the surface of the visor; wherein
the mechanical fastening comprises a fastening portion comprising an array of projections upstanding from the overlay-sheet.

18. A visor comprising a mechanical fastener for attaching an overlay-sheet thereto, wherein the mechanical fastener comprises a fastening portion comprising an array of projections upstanding from the visor.

19. A visor assembly, comprising:
a visor having a surface;
an overlay-sheet;
a mechanical fastening for attaching the overlay-sheet to the surface of the visor; and
a seal member spacing the overlay-sheet from the visor and forming a sealed compartment between the visor and overlay-sheet;
wherein
the mechanical fastening comprises a first fastening portion provided on the surface of the visor, comprising an array of projections upstanding from the visor surface; and
a second fastening portion provided on the overlay-sheet, comprising an array of complementary projections upstanding therefrom; and
wherein the projections of the first and second fastening portions are configured to inter-engage in order to attach the overlay-sheet to the visor, and wherein each projection comprises a stem having a mushroomed portion along its length.

20. A visor assembly, comprising:
a visor having a surface;
an overlay-sheet; and
a mechanical fastening for attaching the overlay-sheet to the surface of the visor;
wherein
the mechanical fastening comprises a first fastening portion provided on the surface of the visor, comprising an array of projections upstanding from the visor surface; and
a second fastening portion provided on the overlay-sheet, comprising an array of complementary projections upstanding therefrom; and
wherein the projections of the first and second fastening portions are adapted to inter-engage in order to attach the overlay-sheet to the visor, and wherein each projection comprises a stem having a mushroomed portion along its length, and wherein the projections are composed of a plastic resin and have a molecular orientation as evidenced by a birefringence value of at least 0.001.
